(19) United States
(12) Patent Application Publication
(10) Pub. No.: US 2025/0130226 A1
(43) Pub. Date: Apr. 24, 2025

(54) SYSTEM AND PROCESS FOR VISUALIZING TARGET MOLECULES

(71) Applicant: LIV PROCESS, INC., Ardmore, PA (US)

(72) Inventors: Ronald J. Shannon, Ardmore, PA (US); Megan A. Touchette, Ardmore, PA (US); Lyndsay M. Pocorobba, Ardmore, PA (US); Michael McIntyre, Ardmore, PA (US)

(73) Assignee: LIV PROCESS, INC., Ardmore, PA (US)

(21) Appl. No.: 18/730,462

(22) PCT Filed: Jan. 12, 2023
(86) PCT No.: PCT/US2023/060547

§ 371 Date: Jul. 19, 2024

Related U.S. Application Data
(60) Provisional application No. 63/301,784, filed on Jan. 21, 2022.

(51) Int. Cl. G01N 33/542; G01N 21/64; G01N 33/569; G01N 33/68

(52) U.S. Cl. CPC G01N 33/542; G01N 21/6447; G01N 21/6456; G01N 33/569; G01N 33/6803

(57) ABSTRACT

Methods and systems of detecting the presence, absence, or amount of target molecule in a sample or on a surface are provided.

### CROSS REFERENCE TO RELATED APPLICATIONS

This application claims the benefit of and the priority to U.S. Provisional Patent Application No. 63/301,784, filed Jan. 21, 2022, the entire disclosure of which is incorporated herein by reference in its entirety.

### TECHNICAL FIELD

Embodiments of the present disclosure relate to systems and methods of detecting the presence, absence, or amount of target molecule in a sample or on a surface.

### SUMMARY

Aspects of the disclosure relate to methods of visualizing presence of one or more pathogen on a surface.

Provided herein are methods of visualizing presence of one or more pathogen on a surface. In some embodiments, the method comprises (a) applying a substantially uniform layer of a liquid composition to the surface, the liquid composition comprising: one or more aptamer conjugate, the one or more aptamer conjugate including one or more aptamer covalently or non-covalently associated with one or more fluorophore, wherein the one or more aptamer conjugate has a specific binding affinity for one or more target molecule of the one or more pathogens; and a quencher to quench nonspecific fluorescence; (b) exposing the surface to a predetermined temperature over a period of time so as to allow a population of conjugated aptamers to bind to the one or more target molecule; (c) illuminating at least one area of the surface by emitting a light at one or more predetermined wavelength range at or around the excitation maximum wavelength of the one or more fluorophore, wherein the one or more fluorophore absorbs the emitted light at the one or more predetermined wavelength range and emits light at a longer wavelength range; and (e) blocking the emitted light, wherein the step of blocking blocks (i) the one or more excitation wavelength range and transmits a second wavelength range that includes some or all of the wavelengths emitted by the one or more fluorophore; or (ii) wavelengths shorter than the emitted wavelength range of the fluorophore.

In some embodiments, the step of applying comprises spraying the surface with the liquid composition. In some embodiments, the step of spraying include a spray distribution with a coefficient of variation (CV) of less than 15%. In some embodiments, the spraying includes minimizing disturbance of the one or more pathogen on the surface. In some embodiments, the step of applying comprises atomizing the liquid composition onto the surface. In some embodiments, the spraying or the atomizing comprises dispersing droplets onto the surface, wherein the droplets have an average size ranging from about 10 to about 100 μm.

In some embodiments, the step of exposing comprises incubating the surface with the one or more aptamer conjugate at room temperature for about 1 min to about 10 min. In some embodiments, the incubating is for less than 5 min.

In some embodiments, the liquid composition comprises two or more conjugated aptamers, wherein each of the two or more aptamer is conjugated to a different fluorophore. In some embodiments, the step of illuminating comprises emitting the light at two or more predetermined wavelength range at or around the excitation maximum wavelength of two or more of the different fluorophores. In some embodiments, the step of illuminating comprises emitting the light at an illumination intensity to enable visualization of the fluorophore at the one or more predetermined wavelength range at or around the excitation maximum wavelength of the one or more fluorophore. In some embodiments, the step of blocking the light comprises using one or more filter, wherein the one or more filter comprises a long pass filter.

In some embodiments, the quencher comprises or consists of graphene oxide.

In some embodiments, the one or more target molecule comprises a protein.

In some embodiments, the liquid composition comprises two or more different aptamers. In some embodiments, the two or more different aptamers bind to different target proteins. In some embodiments, the two or more different aptamers bind to the same target protein.

In some embodiments, the method further comprises imaging the surface.

In some embodiments, the method further comprises identifying the one or more pathogen.

In some embodiments, the method further comprises decontaminating the surface.

In some embodiments, the method further comprises (e) determining the presence of fluorescence from the population of conjugated aptamers or the absence of fluorescence thereby determining the presence or absence of the one or more pathogens onto the surface.

In some embodiments, the method further comprises repeating steps (a) through (e).

In some embodiments, the in step (b) a first population of conjugated aptamers binds to the one or more target; and a second population of conjugated aptamers does not bind to the one or more target molecule and remains associated with the quencher.

In some embodiments, step (c) comprises illuminating the at least one area of the surface with a lighting device having a single emitter that is configured to emit the light at the one or more predetermined wavelength range at or around the excitation maximum wavelength of the one or more fluorophore. In some embodiments, the emitter is configured to emit the light at two or more predetermined wavelength range at or around the excitation maximum wavelength of two or more of the different fluorophores.

In some embodiments, the method comprises (a) applying a substantially uniform layer of a liquid composition to the surface, the liquid composition having: (i) one or more aptamer conjugate, wherein the one or more aptamer conjugate comprises one or more aptamer covalently or non-covalently associated with one or more fluorophore, wherein the one or more aptamer conjugate has a specific binding affinity for one or more target molecule of the one or more pathogens; and (ii) a quencher to quench nonspecific fluorescence; (b) allowing the surface to dry for a period of time at a predetermined temperature, wherein (i) a first population of conjugated aptamers binds to the one or more target molecule; and (ii) a second population of conjugated aptamers not bound to target molecule remains associated with the quencher; (c) illuminating at least one area of the surface with a lighting device that is configured to emit light at one or more predetermined wavelength range at or around the excitation maximum wavelength of the one or more fluorophore, wherein the one or more fluorophore absorbs the light from the lighting device at the one or more predetermined wavelength range and emits light at a longer wavelength range; (d) blocking or filtering out the light from the lighting device using one or more filter configured to substantially (i) block the one or more predetermined excitation wavelength range from the lighting device and transmit a second wavelength range that includes one or more wavelengths emitted by the one or more fluorophore; or (ii) block wavelengths shorter than the emitted wavelength range of the fluorophore; and (e) determining the presence of fluorescence from the first population of conjugated aptamers or the absence of fluorescence thereby determining the presence or absence of the one or more pathogens onto the surface.

In some embodiments, the method does not include a washing the surface before illuminating the surface.

In some embodiments, the method comprises imaging the surface using an imaging device. In some embodiments, the method comprises identifying the one or more pathogen. In some embodiments, the method further comprises decontaminating the surface. In some embodiments, the method further comprises repeating steps (a) through (e).

In some embodiments, the liquid composition is an aqueous composition. In some embodiments, the liquid composition comprises a cocktail of aptamers. In some embodiments, the liquid composition comprises two or more different aptamers. In some embodiments, the one or more aptamers have a binding affinity to one or more target molecule. In some embodiments, the one or more aptamers have a binding affinity to the same target molecule. In some embodiments, the target molecule is a biomolecule. In some embodiments, the biomolecule is a protein. In some embodiments, the target molecule is a protein. In some embodiments, the target molecule is a surface protein. In some embodiments, the target molecule is an antigen. In some embodiments, the target molecule is a surface antigen.

In some embodiment, each aptamer is associated/conjugated to one or more fluorophore. In some embodiment, each aptamer is associated/conjugated to two or more different fluorophores. For example, the fluorophore can comprise but is not limited to FAM, Cy3, Oregon green or any combination of the foregoing.

In some embodiments, the quencher comprises or consists of graphene oxide.

In some embodiments, the applying step comprises spraying the surface. In some embodiments, the step of spraying includes a spray distribution with a coefficient of variation (CV) of less than 15%. In some embodiments, spraying includes minimizing disturbance of the pathogen on the surface. In some embodiments, the spraying is substantially perpendicular to the surface. In some embodiments, the contacting step comprises atomizing the liquid composition onto the surface. In some embodiments, the contacting comprises dispersing droplets onto the surface, wherein the droplets have a size ranging from about 10 to about 100 μm.

In some embodiments, the method comprises incubating the surface with the one or more aptamer conjugate at room temperature for about 1 min to about 10 min, 1 min to 5 min, or less than 5 min.

In some embodiments, the lighting device comprises a single emitter that is configured to emit light at two or more predetermined wavelength range at or around the excitation maximum wavelength of the one or more fluorophore. In some embodiments, the lighting device comprises a single emitter that is configured to emit light at two or more predetermined wavelength range at or around the excitation maximum wavelength of the two or more fluorophores. In some embodiments, the lighting device is configured to switch from a first wavelength to a second different wavelength. In some embodiments, the emitter is configured to emit light at illumination intensity to enable visualization of the fluorophore at the one or more predetermined wavelength range at or around the excitation maximum wavelength of the one or more fluorophore.

In some embodiments, the excitation wavelength can be optimized for a particular fluorophore or fluorescent dye.

In some embodiments, the step of blocking or filtering out the light from the lighting device using one or more filters, wherein the one or more filters are configured to substantially block the excitation wavelength(s) while allowing substantially all of the emission wavelength(s) to pass to allow detection and/or visualization and/or imaging. In some embodiments, the filter is disposed between the surface and the viewer, a detection device or an imaging device to aid for visualization of fluorescence emitted and/or improve the signal to noise ratio. Depending on the fluorophore(s) used, different filters can be used for optimal visualization. For example, if the different fluorophores have sufficiently different emission wavelengths different filters might be required for optimal visualization. In some embodiments, visualization can be performed in ambient light or in a dark environment.

In some embodiments, the one or more filter comprises a long pass filter.

In some embodiments, the one or more filter may be worn as eyewear, for example goggles and eyeglasses. In other embodiments, the filter may be a feature of the imaging device (for example a camera) used to image the surface.

### BRIEF DESCRIPTION OF THE DRAWINGS

Embodiments of the present disclosure will be better understood when read in conjunction with the appended drawings. For the purposes of illustrating the disclosure, there are shown in the drawings embodiments which may be preferred. It is understood, however, that the disclosure is not limited to the precise arrangements and instrumentalities shown.

FIG. 1 is a Table showing the excitation and emission wavelengths and visual colors for non-limiting examples of fluorophores.

FIG. 2 is a non-limiting typical fluorophore absorption-emission spectral diagram.

### DETAILED DESCRIPTION

It is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

As used herein, “aptamer,” “nucleic acid molecule,” or “oligonucleotide” are used interchangeably to refer to a non-naturally occurring nucleic acid molecule that has a desirable action on a target as defined herein. The aptamers described herein are small artificial ligands, comprising DNA, RNA, or modifications thereof, capable of specifically binding to a target as defined herein with high affinity and specificity. In some embodiments, the aptamers may be DNA aptamers. For example, the aptamers may be formed from single-stranded DNA (ssDNA). In some embodiments, the aptamers may be RNA aptamers. For example, the aptamers can be formed from single-stranded RNA (ssRNA). The aptamers of embodiments of the disclosure may comprise modified nucleic acids as described herein.

As used herein the terms “detectable label” and “detectable moiety” are used interchangeably. In some embodiments, the detectable label is a luminescent molecule. In some embodiments, the detectable label is a fluorophore.

As used herein “fluorophores” are molecules with the property of fluorescence. “Fluorescence” is the property of absorbing light of given wavelength and emitting light of longer wavelength.

Aspects of the disclosure relate to methods of visualizing presence of one or more pathogen on a surface. Aspects of the disclosure relate to methods of visualizing presence of one or more pathogen on a surface. In some embodiments, the method comprises (a) contacting the surface with a liquid composition, wherein the contacting results in deposition of a substantially uniform layer of the liquid composition, wherein the liquid composition comprises: (i) one or more aptamer conjugate, wherein the one or more aptamer conjugate comprises one or more aptamer covalently or non-covalently associated with one or more fluorophore, wherein the one or more aptamer conjugate has a specific binding affinity for one or more target molecule of the one or more pathogens; and (ii) a quencher, wherein nonspecific fluorescence is quenched by association with the quencher; (b) allowing the surface to dry for a period of time at a predetermined temperature, wherein (i) a first population of conjugated aptamers binds to the one or more target molecule; and (ii) a second population of conjugated aptamers not bound to target molecule remains associated with the quencher; (c) illuminating at least one area of the surface with a lighting device configured to emit light at one or more predetermined wavelength range at or around the excitation maximum wavelength of the one or more fluorophore, wherein the one or more fluorophore absorb the light from the lighting device at the one or more predetermined wavelength range and emit light at a longer wavelength range; (d) blocking or filtering out the light form the lighting device using one or more filter configured to substantially (i) block the one or more predetermined excitation wavelength range from the lighting device and transmit a second wavelength range that includes one or more wavelengths emitted by the one or more fluorophore; or (ii) block wavelengths shorter than the emitted wavelength range of the fluorophore; and (e) determining the presence of fluorescence from the first population of conjugated aptamers or the absence of fluorescence thereby determining the presence or absence of the one or more pathogens onto the surface.

In some embodiments, the method does not include a washing the surface before illuminating the surface.

In some embodiments, the method comprises imaging the surface using an imaging device. In some embodiments, the method comprises identifying the one or more pathogen. In some embodiments, the method further comprises decontaminating the surface. In some embodiments, the method further comprises repeating steps (a) through (e).

In some embodiments, the liquid composition is an aqueous composition. In some embodiments, the liquid composition comprises a cocktail of aptamers. In some embodiments, the liquid composition comprises two or more different aptamers. In some embodiments, the one or more aptamers have a binding affinity to one or more target molecule. In some embodiments, the one or more aptamers have a binding affinity to the same target molecule. In some embodiments, the target molecule is a biomolecule. In some embodiments, the biomolecule is a protein. In some embodiments, the target molecule is a protein. In some embodiments, the target molecule is a surface protein. In some embodiments, the target molecule is an antigen. In some embodiments, the target molecule is a surface antigen.

In some embodiment, each aptamer is associated/conjugated to one or more fluorophore. In some embodiment, each aptamer is associated/conjugated to two or more different fluorophores. For example, the fluorophore can comprise but is not limited to FAM, Cy3, Oregon green, or any combination of the foregoing.

In some embodiments, the quencher comprises or consists of graphene oxide.

In some embodiments, the method comprises spraying, atomizing, fogging, vaporizing, or coating, immersing, adding an aliquot of the liquid composition directly either by pouring, swabbing, or with a pipetting device. In some embodiments, the liquid composition has a viscosity, ionic strength, and/or pH that can be selected to optimize fluorescence intensity. In some embodiments, the liquid composition has a pH ranging from about 7 to about 8. In some embodiments, the liquid composition comprises a buffer (e.g. Tris buffer) having a pH of about 7.1, 7.2, 7.3, 7.4, 7.5, 7.6, 7.8, 7.9 or 8. In some embodiments, the liquid composition has a ionic strength ranging from about 1 mmol/L (mM) to about 2 mol/L (M). As a non-limiting example, the liquid composition can comprise Tris buffer, magnesium chloride, calcium chloride, sodium chloride, or combination thereof.

In some embodiments, the method comprises depositing or applying a substantially uniform layer of the liquid composition onto a surface. Such a layer, in an embodiment, can also be conformal. In some embodiments, the method comprises spraying the surface. In some embodiments, the spray distribution has a coefficient of variation (CV) of less than about 15%. In some embodiments, the spray distribution has a CV ranging from about 1% to about 15%, from about 1% to about 10%, about 5% to about 15%, from about 5% to about 10%, from about 10% to about 15% or any value therebetween. In some embodiments, the spraying is substantially perpendicular to the surface. In some embodiments, the contacting step comprises atomizing the liquid composition onto the surface. In some embodiments, the contacting step comprises depositing the liquid composition as a fog to disperse droplets and promote better adhesion to the surface, for example on vertical surfaces where the liquid flows downward if droplets size is too big, and volume is too high. In some embodiments, the contacting comprises dispersing droplets onto the surface, wherein the droplets have a size ranging from about 1 to about 300 μm. In some embodiments, the droplets can have an average size of from about 1 to about 50 μm, from about 1 to about 100 μm, from about 1 to about 150 μm, from about 1 to about 200 μm, from about 1 to about 300 μm, from about 10 to about 100 μm, from about 10 to about 150 μm, from about 10 to about 200 μm, from about 10 to about 250 μm, from about 10 to about 300 μm, from about 20 to about 100 μm, from about 20 to about 150 μm, from about 20 to about 200 μm, from about 20 to about 250 μm, from about 20 to about 300 μm, from about 30 to about 100 μm, from about 30 to about 150 μm, from about 30 to about 200 μm, from about 30 to about 250 μm, from about 30 to about 300 μm, from about 40 to about 100 μm, from about 40 to about 150 μm, from about 40 to about 200 μm, from about 40 to about 250 μm, from about 40 to about 300 μm, from about 50 to about 100 μm, from about 50 to about 150 μm, from about 50 to about 200 μm, from about 50 to about 250 μm, from about 50 to about 300 μm, from about 60 to about 100 μm, from about 60 to about 150 μm, from about 60 to about 200 μm, from about 60 to about 250 μm, from about 60 to about 300 μm, from about 70 to about 100 μm, from about 70 to about 150 μm, from about 70 to about 200 μm, from about 70 to about 250 μm, from about 70 to about 300 μm, from about 80 to about 100 μm, from about 80 to about 150 μm, from about 80 to about 200 μm, from about 80 to about 250 μm, from about 80 to about 300 μm, from about 90 to about 100 μm, from about 90 to about 150 μm, from about 90 to about 200 μm, from about 90 to about 250 μm, from about 90 to about 300 μm, from about 100 to about 150 μm, from about 100 to about 200 μm, from about 100 to about 250 μm, from about 100 to about 300 μm, from about 110 to about 150 μm, from about 110 to about 200 μm, from about 110 to about 250 μm, from about 110 to about 300 μm, from about 120 to about 150 μm, from about 120 to about 200 μm, from about 120 to about 250 μm, from about 120 to about 300 μm, from about 130 to about 150 μm, from about 130 to about 200 μm, from about 130 to about 250 μm, from about 130 to about 300 μm, from about 140 to about 150 μm, from about 140 to about 200 μm, from about 140 to about 250 μm, from about 140 to about 300 μm, from about 150 to about 200 μm, from about 150 to about 250 μm, from about 150 to about 300 μm, from about 200 to about 250 μm, from about 250 to about 300 μm. In some embodiments, the droplets can have an average size of from about 1 to about 10 μm, from about 10 to about 20 μm, from about 20 to about 30 μm, from about 30 to about 40 μm, from about 40 to about 50 μm, from about 50 to about 60 μm, from about 60 to about 70 μm, from about 70 to about 80 μm, from about 80 to about 90 μm, from about 90 to about 100 μm, from about 100 to about 110 μm, from about 110 to about 120 μm, from about 120 to about 130 μm, from about 130 to about 140 μm, from about 140 to about 150 μm, from about 150 to about 160 μm, from about 160 to about 170 μm, from about 170 to about 180 μm, from about 180 to about 190 μm, from about 190 to about 200 μm, from about 200 to about 210 μm, from about 210 to about 220 μm, from about 220 to about 230 μm, from about 230 to about 240 μm, from about 240 to about 250 μm, from about 250 to about 260 μm, from about 260 to about 300 μm, from about 270 to about 280 μm, from about 280 to about 290 μm, from about 290 to about 300 μm. For example, the droplets can have an average size of about 1, 2, 3, 4, 5, 6, 7, 8, 9, 10, 20, 30, 34, 50, 60, 70, 80, 90, 100, 110, 120, 130, 140, 150, 160, 170, 180, 190, 200, 210, 220, 230, 240, 250, 260, 270, 280 or 300 μm.

In some embodiments, a fine mist spray pressure of less than <150 psi. can be used to uniformly coat/deposit the liquid composition onto a surface. As noted above, such a coating, in an embodiment, can be conformal.

In some embodiments, a liquid spraying device, also referred herein as sprayer is used to deposit the uniform layer of liquid composition onto the surface. In some embodiments, the sprayer is a handheld device. In some embodiments, the sprayer is a manual sprayer. In some embodiments, the sprayer is a cordless handheld device. In some embodiments, the sprayer is an atomizer. In some embodiments, the sprayer is a nanoatomizer. In some embodiments, the sprayer can provide aerosol functionality. In some embodiments, the sprayer can be configured to pressurize the liquid prior to spaying. See for example U.S. Pat. No. 8,905,271, which is incorporated herein by reference in its entirety. In some embodiments, the liquid can be pressurized to from 500 mbar to 5 bar. In some embodiments, the sprayer is configured to produce a substantially uniform spray with a spray distribution having a coefficient of variation (CV) of less than about 15%. In some embodiments, the sprayer is configured to deliver a continuous spray. In some embodiment, the nozzle of the sprayer can be configured to provide the required discharge and/or droplet size. In some embodiments, the nozzle is configured to provide substantially even distribution of the liquid composition so to result in a substantially uniform coverage of the surface.

It should be appreciated that the spray directed onto the surface to be detected, regardless of the mechanism used, should be dispersed with a force sufficient to minimize disturbance of the pathogen population on the surface to be detected.

In some embodiments, the surface is a vertical or horizontal or inclined surface. In some embodiments, the surface is a surface in a healthcare setting. In some embodiments, the surface is a surface in a food processing setting.

Embodiments comprise methods for detecting the presence, absence, or amount of a target molecule in or on a sample. In the methods, the sample may be interacted (i.e. contacted) with an aptamer conjugate as described herein.

In some embodiments, the method comprises incubating the surface or sample with the one or more aptamer conjugate under conditions sufficient for at least a portion of the aptamer to bind to a target molecule in the sample. In some embodiments, the method comprises exposing the surface to a predetermined temperature over a period of time so as to allow a population of conjugated aptamers to bind to the one or more target molecule. In some embodiments, the method comprises allowing the aptamer(s) to bind the target molecule(s) at room temperature for about 1 min to about 10 min, 1 min to 5 min, or less than 5 min.

A person skilled in the art will understand that the conditions required for binding to occur between the aptamers described herein and a target molecule in a sample or on a surface. In some embodiments, the conjugated aptamer may be incubated at temperatures between about 4° C. and about 40° C. In some embodiments, the sample and aptamer may be incubated at temperatures between about 20° C. and about 37° C. In some embodiments, the sample and aptamer may be incubated at or about 22° C. The incubation temperature may be selected from the range of 4° C. to less than 20° C., 20° C. to less than 22° C., 22° C. to less than 24° C., 24° C. to less than 26° C., 26° C. to less than 28° C., 28° C. to less than 30° C., 30° C. to less than 32° C., 32° C. to less than 34° C., 34° C. to less than 36° C., 36° C. to 37° C., and 37° C. to 40° C. In some embodiments, the sample and aptamer may be diluted to different concentrations (e.g. at least about 1%, 5%, 10%, 20%, 25%, 30%, 40%, 50%, 60%, 70% 80% v/v or more) with water or a buffer (exemplary buffers include but are not limited to Phosphate-buffered saline (PBS), Tris buffer and the like). The diluted concentrations may be selected from the range of 1% to less than 5%, 5% to less than 10%, 10% to less than 20%, 20% to less than 30%, 30% to less than 40%, 40% to less than 50%, 50% to less than 60%, 60% to less than 70%, 70% to less than 80%, or 80% to less than 90%. In some embodiments, the aptamer concentration before dilution may be from 100 nM to 50 μM. In some embodiments, the aptamer concentration before dilution may be selected from the range of 100 nM to 500 nM, 500 nM to 1 μM, 1 μM to 2μ, 2 μM to 5 μM, 5 μM to 10 μM, 10 μM to 15μ, 15 μM to 20 μM, 20 μM to 30 μM, 30 μM to 40 μM, 40 μM to 50 μM, 50 μM to 60 μM, 60 μM to 70 μM, 70 μM to 80 μM, 80 μM to 90 μM, 90 μM to 100 μM. In some embodiments, the aptamer concentration before dilution may be a concentration selected from the ranges described herein in. The selected value may be selected from 0.1 μM increment concentrations in a range herein. In some embodiments, the aptamer concentration before dilution may be 2 μM. In some embodiments, the sample and aptamer may be incubated whilst shaking and/or mixing. In some embodiments, the sample and aptamer are incubated for at least 1 minute, at least 5 minutes, at least 15 minutes, at least 1 hour, or more. The sample and aptamer may be incubated for 1 minute to less than 5 minutes, 5 minutes to less than 15 minutes, 15 minutes to less than one hour, one hour to less than 24 hours, 24 hours to less than 48 hours.

### Conjugated Aptamers

In some embodiments, the aptamers of the disclosure are used to detect and/or quantify the amount of a target molecule in a sample or on a surface. Typically, the aptamers comprise or is conjugated with a detectable label to form a conjugated aptamer. Any label capable of facilitating detection and/or quantification of the aptamers may be used herein. Non-limiting examples of detectable labels are described below.

In some embodiments, the detectable label is a luminescent molecule.

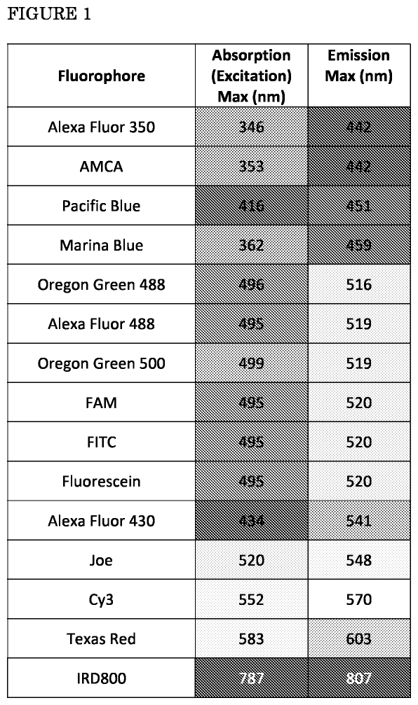

In some embodiments, the detectable label is a fluorescent moiety, e.g. a fluorescent compound (also referred herein as fluorophore) or fluorescent dye. Non-limiting fluorescent dies are shown at FIG. 1. In some embodiments, the aptamer comprises a fluorescent and a quencher compound. Fluorescent and quencher compounds are known in the art. See, for example, Mary Katherine Johansson, Methods in Molecular Biol. 335: Fluorescent Energy Transfer Nucleic Acid Probes: Designs and Protocols, 2006, Didenko, ed., Humana Press, Totowa, NJ, and Marras et al., 2002, Nucl. Acids Res. 30, el22 (incorporated by reference herein).

In some embodiments, the detectable label is a Fluorescein amidite (FAM). In some embodiments, the FAM-label is conjugated to the 5′ end or the 3′ end of the aptamer. One of ordinary skill in the art would understand that the label may be located at any suitable position within the aptamer.

In some embodiments, the aptamer comprises a FAM fluorophore at its 5′ end. In some embodiments, the aptamer is synthesized by incorporating phosphoramidite one at a time into the nucleic acid chain and the FAM-labeled phosphoramidite is incorporated through the synthesis process. In some embodiments, the FAM fluorophore is attached at the 5′ end of the aptamer via a linker. In some embodiments, the detectable label is attached to an aptamer described herein via a moiety selected from a thiol group, an amine group, an azide, six-carbon linker, and an aminoallyl group and combinations thereof. In some embodiments, the FAM label can be incorporated into the aptamer using a forward primer with a FAM on the 5′ end. In some embodiments, the aptamer can be prepared by solid phase synthesis with the FAM label already in place, attached to the 5′ end as in the primer.

Moieties that result in an increase in detectable signal when in proximity of each other may also be used herein, for example, as a result of fluorescence resonance energy transfer (“FRET”); suitable pairs include but are not limited to fluorescein and tetramethylrhodamine; rhodamine 6G and malachite green, and FITC and thiosemicarbazole, to name a few.

In some embodiments, the detectable label is and/or comprises a moiety selected from at least one of the following non-limiting examples: a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

In some embodiments, the detectable label is a fluorescent protein such as Green Fluorescent Protein (GFP) or any other fluorescent protein known to those skilled in the art.

In some embodiments, the detectable label is an enzyme. For example, the enzyme may be selected from horseradish peroxidase, alkaline phosphatase, urease, β-galactosidase or any other enzyme known to those skilled in the art.

In some embodiments, the nature of the detection will be dependent on the detectable label used. For example, the label may be detectable by virtue of its color e.g. gold nanoparticles. A color can be detected quantitatively by an optical reader or camera e.g. a camera with imaging software.

In some embodiments, the detectable label is a fluorescent label e.g. a quantum dot. In such embodiments, the detection means may comprise a fluorescent plate reader, strip reader or similar, which is configured to record fluorescence intensity.

In some embodiments in which the detectable label is an enzyme label, non-limiting detection means may, for example, be colorimetric, chemiluminescence and/or electrochemical (including, but not limited to using an electrochemical detector). Electrochemical sensing may be through conjugation of a redox reporter (including, but not limited to methylene blue or ferrocene) to one end of the aptamer and a sensor surface to the other end. A change in aptamer conformation upon target binding may change the distance between the reporter and sensor to provide a readout.

In some embodiments, the detectable label may further comprise enzymes, including but not limited to, horseradish peroxidase (HRP), Alkaline phosphatase (APP) or similar, to catalytically turnover a substrate to give an amplified signal.

Embodiments comprise a complex (e.g. conjugate) comprising aptamers of the disclosure and a detectable molecule. Typically, the aptamers of the disclosure are covalently or physically conjugated to a detectable molecule.

In some embodiments, the detectable molecule is a visual, optical, photonic, electronic, acoustic, opto-acoustic, mass, electrochemical, electro-optical, spectrometric, enzymatic, or otherwise physically, chemically or biochemically detectable label.

In some embodiments, the detectable molecule is detected by luminescence, UV/VIS spectroscopy, enzymatically, electrochemically or radioactively. Luminescence refers to the emission of light. For example, photoluminescence, chemiluminescence and bioluminescence are used for detection of the label. In photoluminescence or fluorescence, excitation occurs by absorption of photons. Exemplary fluorophores include, but are not limited to, bisbenzimidazole, fluorescein, acridine orange, Cy5, Cy3 or propidium iodide, which can be covalently coupled to aptamers, tetramethyl-6-carboxyrhodamine (TAMRA), Texas Red (TR), rhodamine, Alexa Fluor dyes. Fluorescent dyes that are excited and emit different wavelengths of light have been developed from different companies.

In some embodiments, the detectable molecule is a colloidal metallic particle, including but not limited to a gold nanoparticle, colloidal non-metallic particle, quantum dot, organic polymer, latex particle, nanofiber (carbon nanofiber, as a non-limiting example), nanotube (carbon nanotube, as a non-limiting example), dendrimer, protein, or liposome with signal-generating substances. Colloidal particles may be detected colorimetrically.

In some embodiments, the detectable molecule is an enzyme. In some embodiments, the enzyme may convert substrates to colored products. Examples of the enzyme include but are not limited to peroxidase, luciferase, B-galactosidase or alkaline phosphatase. For example, the colorless substrate X-gal is converted by the activity of 8-galactosidase to a blue product whose color is visually detected.

In some embodiments, the detection molecule is a radioactive isotope. The detection may also be carried out by means of radioactive isotopes with which the aptamer is labelled, including but not limited to ³H, ¹⁴C, ³²P, ³³P, ³⁵S or ¹²⁵I. In some embodiments, scintillation counting may be conducted, and thereby the radioactive radiation emitted by the radioactively labelled aptamer target complex is measured indirectly. A scintillator substance is excited by the isotope's radioactive emissions. During the transition of the scintillation material, back to the ground state, the excitation energy is released again as flashes of light, which are amplified and counted by a photomultiplier.

In some embodiments, the detectable molecule is selected from digoxigenin and biotin. Thus, the aptamers may also be labelled with digoxigenin or biotin, which are bound for example by antibodies or streptavidin, which may in turn carry a label, such as an enzyme conjugate. The prior covalent linkage (conjugation) of an aptamer with an enzyme can be accomplished in several known ways.

In some embodiments, detection of aptamer binding may also be achieved through labelling of the aptamer with a radioisotope in an RIA (radioactive immunoassay), preferably with ¹²⁵I, or by fluorescence in a FIA (fluoroimmunoassay) with fluorophores. In some embodiments, the fluorophore is fluorescein or fluorescein isothiocyanate (FITC).

In some embodiments, the detectable moiety comprises a fluorescent moiety and visualization comprises visualizing and/or measuring the level of fluorescence. In some embodiments, the detectable moiety comprises biotin having a binding affinity for streptavidin protein conjugates, such as streptavidin/horseradish peroxidase and visualization comprises visualizing using a colorimetric reaction. In some embodiments, the detectable moiety gold nanoparticles conjugated to the aptamer and visualization comprises visualizing using a colorimetric assay. In some embodiment, the detectable moiety comprises a quantum dot, that fluoresces.

In some embodiments, binding of the aptamer and a target as defined leads to formation of an aptamer-target complex. The binding or binding event may be detected, for example, visually, optically, photonically, electronically, acoustically, opto-acoustically, by mass, electrochemically, electro-optically, spectrometrically, enzymatically or otherwise chemically, biochemically, or physically as described herein.

The binding of aptamer and the target may be detected using any suitable technique. As discussed above, for example, binding of the aptamer and the target may be detected using a biosensor. In some embodiments, binding of the aptamer and the target is detected using the non-limiting examples of SPR, RIfS, BLI, LFD or ELONA as described herein.

In some embodiments, the aptamer can be attached to the surface of the biosensor using a biotin group. In some embodiments, the biotin group is attached at the 5′ end or the 3′ end of the aptamer. In some embodiments, the surface of the biosensor has an avidin/streptavidin attached thereto and the immobilization of the aptamer to the surface of the biosensor is via biotin-avidin interaction. In some embodiments, the surface of the biosensor is coated with avidin/streptavidin.

In some embodiments, the aptamer is linked or conjugated to a fluorescent moiety. In some embodiments, the aptamer is an aptamer conjugate comprising an aptamer conjugated with a fluorescent moiety. In some embodiments the fluorophore is at the 5′ end or the 3′ end of the aptamer. In some embodiments, the aptamer is associated with an antisense oligonucleotide having a fluorophore. In some embodiments the fluorophore is at the 5′ end or the 3′ end of the aptamer. In some embodiments the antisense oligonucleotide is complementary to the 5′ end or 3′ end of the aptamer. In some embodiments, the fluorophore is at the 5′ end or the 3′ end of the antisense oligonucleotide.

In some embodiments, the aptamers of the disclosure are prepared using principles of in vitro selection known in the art, that include iterative cycles of target binding, partitioning and preferential amplification of target binding sequences. Selection may be performed using immobilized target proteins. Immobilization may include, but is not limited to, immobilization to a solid surface. In a non-limiting example, the solid surface may be beads. In a non-limiting example, the solid surface may be magnetic beads.

Non-limiting examples of amplification methods include polymerase chain reaction (PCR), ligation amplification (or ligase chain reaction, LCR), strand displacement amplification, nucleic acid sequence-based amplification, and amplification methods based on the use of Q-beta replicase. In a non-limiting embodiment, at least one type of aptamer may be immobilized on a solid surface during amplification. Each of these exemplary methods is well known in the art.

In some embodiments, the aptamers are selected from a nucleic acid molecule library such as a single-stranded DNA or RNA nucleic acid molecule library. The aptamers may be selected from a “universal aptamer selection library” that is designed such that any selected aptamers need little to no adaptation to convert into any of the listed assay formats.

Once selected, the aptamer may be further modified before being used e.g. to remove one or both primer sequences and/or parts of the randomized sequence not required for target binding.

Typically, aptamers of the embodiments of the disclosure comprise a first primer region (e.g. at the 5′ end), a second primer region (e.g. at the 3′ end), or both. The primer regions may serve as primer binding sites for PCR amplification of the library and selected aptamers.

The first primer region and/or second region may comprise a detectable label as described herein. As used herein the terms “detectable label” and “detectable moiety” are used interchangeably. In some embodiments, the first and/or second primer region may be labelled with a luminescent molecule. In some embodiments, the first and/or second primer region may be fluorescently labelled. Non-limiting examples of fluorescent labels include but are not limited to fluorescein, green fluorescent protein (GFP), yellow fluorescent protein, cyan fluorescent protein, and others. In some embodiments, a fluorescein label is used. In some embodiments, other forms of detecting the primer may be used, including but not limited to phosphate (PO₄) labelling, isotope labelling, electrochemical sensors, colorimetric biosensors, and others.

The aptamers may comprise natural or non-natural nucleotides and/or base derivatives (or combinations thereof). In some embodiments, the aptamers comprise one or more modifications such that they comprise a chemical structure other than deoxyribose, ribose, phosphate, adenine (A), guanine (G), cytosine (C), thymine (T), or uracil (U). The aptamers may be modified at the nucleobase, at the sugar or at the phosphate backbone.

In some embodiments, the aptamers comprise one or more modified nucleotides. Exemplary modifications include for example nucleotides comprising an alkylation, arylation or acetylation, alkoxylation, halogenation, amino group, or another functional group. Examples of modified nucleotides include, but are not limited to, 2′-fluoro ribonucleotides, 2′—NH₂—, 2′—OCH₃— and 2′-O-methoxyethyl ribonucleotides, which are used for RNA aptamers.

The aptamers may be wholly or partly phosphorothioate or DNA, phosphorodithioate or DNA, phosphoroselenoate or DNA, phosphorodiselenoate or DNA, locked nucleic acid (LNA), peptide nucleic acid (PNA), N3′-P5 'phosphoramidate RNA/DNA, cyclohexene nucleic acid (CeNA), tricyclo DNA (tcDNA) or spiegelmer, or the phosphoramidate morpholine (PMO) components or any other modification known to those skilled in the art (see also Chan et al., Clinical and Experimental Pharmacology and Physiology (2006) 33, 533-540).

Some of the modifications may allow the aptamers to be stabilized against nucleic acid-cleaving enzymes. In the stabilization of the aptamers, a distinction can generally be made between the subsequent modification of the aptamers and the selection with already modified RNA/DNA. The stabilization may not affect the affinity of the modified RNA/DNA aptamers but may prevent the rapid decomposition of the aptamers in an organism, biological solutions, or solutions, by RNases/DNases. An aptamer is referred to as stabilized if the half-life of the aptamer in the sample (e.g. biological medium, organism, solution) is greater than one minute, greater than one hour, or greater than one day. The aptamers may be modified with reporter molecules, which may enable detection of the labelled aptamers. Reporter molecules may also contribute to increased stability of the aptamers.

Aptamers form a three-dimensional structure that depends on their nucleic acid sequence. The three-dimensional structure of an aptamer may arise due to Watson and Crick intramolecular base pairing, Hoogsteen base pairing (quadruplex), wobble-pair formation, or other non-canonical base interactions. In some embodiments, the three-dimensional structure enables aptamers, analogous to antigen-antibody binding, to bind target structures accurately. A nucleic acid sequence of an aptamer may, under defined conditions, have a three-dimensional structure that is specific to a defined target structure.

Embodiments comprise competitive aptamers that compete for binding to a target protein as defined herein with aptamers as described herein. Embodiments comprise competitive one or more aptamers that compete for binding to a target protein as defined herein with one or more of the aptamers described above. In some embodiments, competition assays may be used identify a competitive aptamer that competes for binding to a target protein as defined herein. In an exemplary, non-limiting, competition assay, an immobilized target protein as defined herein is incubated in a solution comprising a first labelled aptamer that binds to a target protein as defined herein and a second unlabeled aptamer that is being tested for its ability to compete with the first aptamer for binding to a target protein as defined herein. As a control, an immobilized target protein as defined herein may be incubated in a solution comprising the first labelled aptamer but not the second unlabeled aptamer. After incubation under conditions permissive for binding of the first aptamer to a target protein as defined herein excess unbound aptamer may be removed, and the amount of label associated with immobilized target protein as defined herein measured. If the amount of label associated with immobilized target as defined herein is substantially reduced in the test sample relative to the control sample, then that indicates that the second aptamer is competing with the first aptamer for binding to a target protein as defined herein.

Full length aptamer sequences have the capacity to form multiple three-dimensional configurations (also referred herein as “shape”) at room temperature. These three-dimensional configurations are in flux, based on an energy landscape among possible configurations. Structural elements of the aptamers are responsible for binding to the target. In some embodiments, truncation of the aptamer sequences and changing of the sequences to stabilize the structural elements responsible for binding can improve aptamer performance by reducing the energy landscape of the optimized aptamer such that only the configuration that binds to the target is present, or at least by increasing the probability of such configurations (and thus presence over time).

In some embodiments, the aptamers are modified to form secondary and/or tertiary conformation to improve the binding affinity of the aptamer to the target molecule.

It should be appreciated that a given aptamer may exist in dynamic equilibrium among many possible shapes or conformations. These structures can be in flux amongst each other. The binding affinity of the aptamer to the target protein is dependent on the structure of the aptamers. In some embodiments, the aptamer structure comprises one or more stem and loop.

To optimize binding effectiveness of a given structure to a target protein, it is desirable if the structure of the selected aptamer is not in flux with other structures (for example in different environments) but is the structure which is predominantly present. As such, although the aptamers are selected using an affinity-based selection assay as described herein, further optimization may be required to achieve the desired binding affinity to the target protein. The predicted conformation(s)/structure(s) of each aptamer can be obtained in silico from the primary sequence. In some embodiments, the primary structure of the aptamers can be engineered (e.g. substitution, deletion) to stabilize the secondary structures or tertiary structures. In some embodiments, the aptamers can be truncated to stabilize the secondary structures.

In some embodiments the aptamers are selected using an affinity-based selection assay, the predicted conformations is obtained in silico, the primary sequence is optimized (e.g. truncation/deletion, substitutions, etc.) so that the optimized aptamer exhibits the optimized conformation and is stabilized. The resulting optimized aptamers have fewer structures that are in flux or exhibit a range or difference among structures in flux that is less than the non-optimized aptamers. These optimized aptamers can be retested for binding effectiveness in order to determine whether the structure that was stabilized is the desired structure that binds to the target protein.

An aptamer may have a secondary structure having at least two complementary regions of the same nucleic acid strand that base-pair to form a double helix (referred to herein as a “stem”). A stem as described herein may be referred to by the position, in a 5′ to 3′ direction on the aptamer, of the 5′ side of the stem (i.e., the stem sequence closer to the 5′ terminus of the aptamer), relative to the 5′ side of additional stems present on the aptamer.

For example, stem 1 may refer to the stem sequence that is closest to the 5′ terminus of the aptamer, its complementary stem sequence, or both stem sequences collectively. Similarly, stem 2 may refer to the next stem sequence that is positioned 3′ relative to stem 1, its complementary stem sequence, or both stem sequences collectively. In some cases, the aptamers of the disclosure have one or more stems. For example, the aptamers of the disclosure can have 1, 2, 3 or ore stems. Each additional stem may be referred to by its position, in a 5′ to 3′ direction, on the aptamer, as described above. For example, stem 2 may be positioned 3′ relative to stem 1 on the aptamer, stem 3 may be positioned 3′ relative to stem 2 on the aptamer, and so on. A stem may be adjacent to an unpaired region. An unpaired region may be present at a terminus of the aptamer or at an internal region of the aptamer.

A stem as described herein may be referred to by its position in a 5′ to 3′ direction on the aptamer. A stem as described herein may be referred to by its length (1, 2, 3 4, 5, 6 or more base pairs). For example, stem (4f) refers 5′ side of a 4 base pairs stem structure. Stem (4r) refers 3′ side of a 4 base pairs stem structure.

As used herein, the term “loop” generally refers to an internal unpaired region of an aptamer. The term “loop” generally refers to any unpaired region of an aptamer that is flanked on both the 5′ end and the 3′ end by a stem region. In some cases, a loop sequence may be adjacent to a single base-paired stem, such that the loop and stem structure together resemble a hairpin. In such cases, generally the primary sequence of the aptamer contains a first stem sequence adjacent to the 5′ end of the loop sequence and a second stem sequence adjacent to the 3′ end of the loop sequence; and the first and second stem sequences are complementary to each other.

A loop as described herein may be referred to by its position in a 5′ to 3′ direction on the aptamer. A loop as described herein may be referred to by its length (1, 2, 3 4, 5, 6 or more nucleotides). For example, a loop (4) refers to a loop structure having 4 nucleotides.

The term “stem-loop” as used herein generally refers to the secondary structure of an aptamer of the disclosure having at least one stem and at least one loop. In some cases, a stem-loop secondary structure includes structures having two stems, which may include a terminal stem, an internal loop, an internal stem, and a terminal loop. A “terminal stem” as used herein generally refers to a stem that encompasses both the 5′ and/or 3′ terminus of the aptamer. In some cases, a “terminal stem” is bordered at one or both termini by a “tail” comprising one or more unpaired nucleotides. For example, a terminal stem present in the aptamer may be bordered by a tail of one or more unpaired nucleotides (or other structures) at its 5′ end. Similarly, a terminal stem present in the aptamer may be bordered by a tail of one or more unpaired nucleotides (or other structures) at its 3′ end. In some cases, a stem-loop secondary structure includes structures having more than two stems. Unless otherwise stated, when an aptamer includes more than one stem and/or more than one loop, the stems and loops are numbered consecutively in ascending order from the 5′ end to the 3′ end of the primary nucleotide sequence.

In some embodiments, the aptamer is an aptamer beacon that undergoes a conformational change when the aptamer binds to the target protein and the detection of the binding of the aptamer to the target protein relies on the conformational change of the aptamer.

In some embodiments, the aptamer conjugate is an aptamer comprising a fluorescent moiety at a first end of the aptamer and a quencher moiety at a second end of the aptamer. In some embodiments, the aptamer comprises a loop, a first nucleic acid segment that is complementary to a second nucleic acid segment, wherein the first segment and the second segment forms a stem portion when the first segment and the second segment are hybridized, wherein the first segment of the aptamer comprises a fluorophore and the second segment of the aptamer comprises a quencher.

In some embodiments, antisense oligonucleotides can be designed to hybridize to the first segment, the second segment or combination thereof and to disrupt the stem and loop structure of the aptamers. For example, the antisense oligonucleotides can be complementary to the 5′ end, the 3′ end, the 5′ end and the 3′ end of or any relevant sequence of the aptamer. In some embodiments, two antisense oligonucleotides are provided, wherein the first antisense oligonucleotide comprises a fluorophore and hybridizes to the first segment of the aptamer, the second antisense comprises a quencher and hybridizes to the second segment of the aptamer.

In some embodiments, the quencher comprises a “dark” quencher. In some embodiments, the quencher comprises a Black Hole Quencher® (BHQ). For example, the 3 'end of the antisense oligonucleotides can be linked to a Black Hole Quencher®.

In some embodiments, the antisense oligonucleotides act competitively with the binding of the aptamer to the target protein.

In some embodiments, upon binding of the aptamer to the target protein, the aptamer undergoes a conformation change, altering the distance between the fluorophore and the quencher, resulting in the emission of a fluorescent signal.

In some embodiments, two or more different aptamers are provided configured to bind to two or more different target proteins in a sample, each aptamer comprising a different fluorophore.

### Graphene Oxide

In some embodiments, a composition comprising one or more aptamer conjugates and graphene oxide (GO) is provided. Graphene oxide self-assembles into two-dimensional sheets in an aqueous environment (See He et al., Chemical Physical Letters, Volume 287, Issues 1-2, 24 Apr. 1998, Pages 53-56). In some embodiments, the composition is a suspension. In some embodiments, the composition comprises a buffer. In some embodiments, the composition further comprises a blocking agent to minimize or eliminate non-specific binding. In some embodiments, the blocking agent includes, but is not limited to, polyethylene glycol (PEG) (including polymeric chain of various lengths), Tween (e.g., Tween 20, Tween 40, Tween 80), nucleic acid (e.g. oligonucleotides, sheared salmon sperm DNA), polyvinyl pyrrolidine or any blocking agent known in the art.

In some embodiments, are provided fluorescently labeled aptamers in combination with graphene oxide to enable surface detection of target molecules. Without being bound to any particular theory, the labeled aptamers bind to graphene oxide (GO) by balancing binding through Π-Π stacking and hydrogen bonding with electrostatic repulsion. The proximity of the fluorophore label on the aptamer to the GO surface results in fluorescence quenching. The introduction of a mixture of aptamer and graphene oxide to a target molecule or a sample comprising a target molecule that the aptamer binds to results in a change in the equilibrium formed between the aptamer and GO, such that less aptamer is bound to GO, and more aptamer is bound to the target. The fluorescence of the aptamer bound to the target is not quenched, or at least not quenched to the same degree that it was when the aptamer was bound to GO.

In some embodiments, this aptamer/GO mixture can be used for the detection of a visual change on a surface. In some embodiments, no fluorescence is visible/detected when an aptamer/GO equilibrated mixture is sprayed onto a surface where no target molecule is present. In some embodiments, fluorescence is observed/detected in the presence of the target molecule (or pathogens).

An equilibrated mixture of aptamer/GO refers to a mixture of aptamer/GO having a relative fluorescence that does not change over time. In some embodiments, this equilibration can be achieved less than 24 hours after the aptamer(s) and GO are mixed. For example, the equilibration can be achieved in a about or less than 5 min, 10 min, 20 min, 30 min., 40 min. 50 min., h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h 22 h, 23 h or 24 h.

In some embodiments, the composition comprises one or more different aptamers having a binding affinity to the same target molecule. In some embodiments, the composition comprises one or more different aptamers having a binding affinity to one or more different target molecules. In some embodiments, each aptamer can comprise a different detectable moiety.

Graphene oxide is prepared from graphene by the exposure of graphene to oxygen donor sources such as NaNO₃, H₂SO₄, H₃PO₄ and KMnO4. In some embodiments, the graphene oxide comprises an oxygen content of about 36%. In some embodiments, the graphene oxide comprises an oxygen content greater than 36%. In some embodiments, the graphene oxide comprises an oxygen content of about 44-45%.

The graphene oxide once formed self-assembles into two-dimensional sheets of varying sizes. In some embodiments, the total surface area of the graphene oxide is about 736.6 m²/g. In some embodiments, the total surface area of the graphene oxide is a function of the amount of graphene oxide used. In some embodiments, the amount of graphene oxide used is optimized based on the level of fluorescence quenching. Without being bound to the theory, determination of the appropriate amount of GO to be used for a desired level of aptamer quenching can be a consideration of the total surface area of the GO in the solution. In some embodiments, the workable range is defined as a function of the desired quenching range, given the need to visualize fluorescence in the presence of the virus and not in the absence.

In some embodiments, the graphene oxide is in the form of nanoparticles. In some embodiments, the average size of the nanoparticles is 10 to 500 nm.

Aptamers (APT) adhere to the graphene oxide (GO) sheets based on Van der Waals forces and hydrogen bonds. In some embodiments, when a detectable moiety such as a fluorescent moiety is conjugated to the aptamer, the fluorescence of the fluorescent moiety is quenched by the association with the graphene oxide surface.

In some embodiments, in the presence of a target surface protein for which the aptamer binds with a binding affinity greater than binding affinity to the graphene oxide, the aptamer is “displaced” from the graphene oxide surface and becomes bound to the target protein. In some embodiments, the aptamer binds with a binding affinity that is twice, ten, twenty, thirty, forty, fifty, sixty, seventy, eighty, ninety, hundred greater, five hundred, thousand times or more than binding affinity to the graphene oxide. In some embodiments, the aptamer binds with a binding affinity that is between 2 and 1000-fold, between 10- and 1000-fold, between 50- and 1000-fold, between 100- and 1000-fold, between 2- and 100-fold, between 10- and 100-fold, between 50- and 100-fold, between 100- and 200-fold, between 100- and 500-fold, between 100- and 1,000-fold, than binding affinity to the graphene oxide. When the aptamer is “displaced” from the graphene due to its specific binding to the target protein, fluorescence is emitted. A minimum of two times, the most preferred enablement would be 100-fold, an acceptable range would be between 10- and 1000-fold.

Without being bound to the theory, in physical terms, the aptamer is not displaced (dynamically binding and unbinding rapidly) and a relationship between the aptamer and the graphene oxide surface can be described by the following linked differential equations.

$$\begin{matrix}Dx 1 = - a * x 1 * x 2 + b * x 3 \\ Dx 2 = - a * x 1 * x 2 + b * x 3 \\ Dx 3 = a * x 1 * x 2 - b * x 3\end{matrix}$$

wherein x1 corresponds to the concentration of aptamer, x2 corresponds to the concentration of graphene oxide, x3 corresponds to the concentration of the complex aptamer-graphene oxide and “D” stands for derivative as a function of time. D is the derivative of their concentrations over time, and ‘a’ and ‘b’ are the on and off binding rates for complex formation.

These equations describe the movement of the system towards an equilibrium. At equilibrium, the system is not static, the molecules are still associating and disassociating at the same rate, it is just that the overall measurement of the system results in no changes in expression of fluorescence.

As such, the introduction of a further element [T] for concentration of a target protein that the aptamer binds to, displaces this equilibrium such that the concentration of Aptamer/GO is decreased in relation to the amount of Aptamer/T formed.

As such, a new system of equations can be elaborated as follows:

$$\begin{matrix}Dx 1 = - a * x 1 * x 2 + b * x 3 - c * x 1 * x 4 + d * x 5 \\ Dx 2 = - a * x 1 * x 2 + b * x 3 \\ Dx 3 = a * x 1 * x 2 - b * x 3 \\ Dx 4 = - c * x 1 * x 4 + d * x 5 \\ Dx 5 = c * x 1 * x 4 - d * x 5\end{matrix}$$

wherein x1 corresponds to the concentration of aptamer, x2 corresponds to the concentration of graphene oxide, x3 corresponds to the concentration of the complex aptamer-graphene oxide, x4 corresponds to the concentration of target, x5 corresponds to the concentration of the complex between the free target and the aptamer, ‘c’ and ‘d’ are the on and off rates for complex formation between the aptamer and the target.

When d/c is a lower number than b/a, equilibrium will shift towards a higher amount of x5 and a lower amount of x3 resulting in the expression of fluorescence.

In some embodiments, the methods provided herein allow for a limit of detection of 25 nM or more.

In some embodiments, the aptamer to target protein ratio may play a role in enhancing the signal at lower concentrations. Without being bound to the theory, this is because at lower concentrations of aptamer, a lower amount of target protein may be needed to make a significant change in recovery (or proportional change). In some embodiments, using lower amount of aptamer may help in detecting concentration lower than about 25 nM, for example about 10 pM or about 100 fM.

In some embodiments, there is provided a method of detecting the presence or absence of a target molecule comprising:
Providing one or more aptamer conjugates comprising one or more aptamers conjugated to one or more detectable moiety.
Combining the aptamer conjugates with a pre-determined concentration of graphene oxide;
Contacting the aptamer conjugates-graphene oxide combination with a sample to be tested;
Visualizing the detectable moiety of the aptamer conjugate bound to the target molecule.

In some embodiments, the method further comprises the step of incubating the aptamer conjugates with the sample for a predetermined period of time to allow the aptamer conjugate to bind to the target molecule if present.

In some embodiments, the sample is contacted first with a blocking agent and subsequently with a combination of aptamer conjugates-graphene oxide.

In some embodiments, the visualizing step comprises imaging the sample. In some embodiments, the visualizing step comprises measuring the level of fluorescence. In some embodiments, the visualizing step comprises comparing the fluorescence level to a negative control sample. In some embodiments, the method does not comprise a washing step before the visualizing step.

### Visualization

In some embodiments, the method comprises illuminating a location (e.g. an area of a surface or the sample) with a lighting device. In some embodiments, the lighting device used is configured to emit light at a wavelength or wavelength range so as to allow visualization of the detectable molecule conjugated to the aptamer. In some embodiments, the detection molecule is a fluorophore that absorbs and is excited by the wavelength(s) output of the lighting device.

In some embodiments, the lighting device is configured to emit light at a predetermined wavelength range, wherein the predetermined wavelength corresponds to a wavelength of light that will be absorbed and subsequently excite the detectable moiety of the aptamer conjugate. For example, the aptamer may be conjugated to a fluorophore which absorbs a wavelength of about 505 nm and the lighting device produces light having a wavelength of about 506 nm.

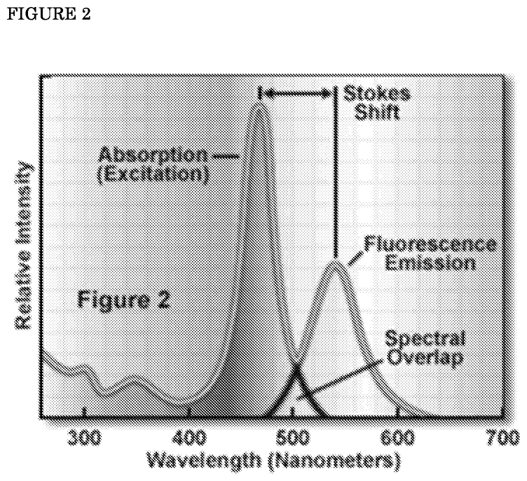

A typical fluorophore absorption-emission spectral diagram is illustrated in FIG. 2. Fluorophores are photoreactive chemical compounds that absorb light energy of a certain wavelength and emit that light at a longer wavelength. Because of their novel electronic configurations, fluorophores have unique and characteristic spectra for absorption (excitation) and emission. These absorption and emission spectra show relative intensity of fluorescence, with the relative intensity classically plotted on the vertical axis versus wavelength on the horizontal axis. For a given fluorophore, the manufacturers indicate the wavelength for the peak of the illumination excitation intensity (excitation maximum) and the wavelength for the peak of fluorescence emission intensity (emission maximum). In some embodiments, when a fluorophore is conjugated to an aptamer, the excitation and emission spectra shifts to lower or higher excitation or emission wavelengths.

In some embodiments, the lighting device can have a single light emitter. In some embodiments, the lighting device can have a plurality of identical light emitters, for example to increase brightness. In some embodiments, a plurality of lighting devices having the identical light emitters can be used, for example to increase brightness. In some embodiments, the emitter is a light emitting diode (LED). In some embodiments, the emitter is a laser or the like.

In some embodiments, the single light emitter is configured to emit light at a single predetermined wavelength. In some embodiments, the single light emitter is configured to emit light at a single predetermined range of wavelengths. In some embodiments, the single predetermined wavelength or single predetermined range of wavelengths is at or around the excitation maximum wavelength of the one or more fluorophore. For example, the range of wavelengths has a peak wavelength at or around the excitation maximum wavelength of the one or more fluorophore. In some embodiments, the single light emitter is configured to emit light at two or more predetermined wavelength or two or more predetermined wavelength ranges. In some embodiments, the two or more predetermined wavelengths or two or more predetermined ranges of wavelengths are at or around the excitation maximum wavelength of the one or more fluorophore. In some embodiments, the two or more predetermined wavelengths or two or more predetermined ranges of wavelengths are at or around the excitation maximum wavelength of the two or more fluorophores.

For example, the emitter may emit light within a specified wavelength range or may be engineered to produce any range of light from about 350 nm to 740 nm. For example, in some embodiments, the emitter may emit light in a wavelength range from about 360 nm to 385 nm (UV light). In some embodiments, the emitter may produce light at a wavelength of between about 405 nm-420 nm. In some embodiments, the emitter may produce light at a wavelength of between about 435 nm-465 nm. In some embodiments, the emitter may produce light at a wavelength of between about 485 nm-515 nm. In some embodiments, the light source may produce light at a wavelength of between about 490 nm-505 nm. In some embodiments, the emitter may produce light at a wavelength of between about 510 nm-545 nm. In some embodiments, the emitter may produce light at a wavelength of between about 530 nm-560 nm. In some embodiments, the emitter may produce light at a wavelength of between about 685 nm-605 nm. In some embodiments, the emitter may produce light at a wavelength of between about 615 nm-635 nm. In some embodiments, the emitter may produce light at a wavelength of between about 400 nm-700 nm. In some embodiments, the emitter may produce light at a wavelength of between about 835 nm-865 nm. In some embodiments, the emitter may produce light at a wavelength of between about 935 nm-965 nm. In some embodiments, the emitter is configured to produce narrow bands of light having center wavelengths of 365 nm, 415 nm, 450 nm, 485 nm, 505 nm, 530 nm, 545 nm, 620 nm, and 850 nm. In some embodiments, the emitter is configured to produce an exact band of light at 485 nm with a +/−2% variance. In some embodiments, the emitter is configured to produce narrow bands of light having a center wavelength of 505 nm.

In some embodiments, the single emitter is configured to pulse/switch from a first predetermined wavelength to a second predetermined wavelength.

In some embodiments, the excitation wavelength can be optimized for a particular fluorophore or fluorescent dye. Depending on the fluorophore, the excitation wavelength can range from about 350 nm to about 650 nm (including, but not limited to from about 440-460 nm, from about 490 to 515 nm, about 505 nm, about 480 nm etc). Depending on the fluorophore, the excitation wavelength can range from about 450 nm to about 670 nm, the emission wavelength being longer than the excitation wavelengths as depicted in FIG. 1 and FIG. 2. In some embodiments, the excitation wavelength range for a FAM fluorophore could include but is not limited to 475 to 515 nm, the excitation wavelength range for a Cy3 fluorophore could include but is not limited to 490 to 560 nm. In some embodiments, the lighting device is configured to emit a light at a wavelength or wavelength range close to the maximum excitation wavelength of the fluorophore so to allow visualization of the output fluorescence. For example, the maximum excitation wavelength for FAM is excitation wavelength of 495 nm and the lighting device configured to visualize FAM-aptamer conjugate can have a wavelength ranging from about 485 nm to about 515 nm. In some embodiments, the emitter can generate light of sufficient illumination intensity to enable visualization of the fluorophore at a predetermined wavelength or predetermined range of wavelengths. In some embodiments, the emitter has an output power (or wattage) sufficient to enable visualization of the fluorophore at a predetermined wavelength or predetermined range of wavelengths.

In some embodiments, the lighting device is configured to emit light at a predetermined lumen value or range of values that allow visualization of the aptamer conjugate. In some embodiments, the lighting device is configured to provide between about 4500 and about 6500 lumens. For example, the lighting device is for visualizing a FAM-aptamer conjugate can be configured to provide about 5500 lumens.

In some embodiments, the lighting device is configured to emit light at a predetermined lux value or range of values that allow visualization of the aptamer conjugate. In some embodiments, the lighting device is configured to provide an intensity at 505 nm of about 200,000 to 280,000 in Lux. For example, the lighting device for visualizing a FAM-aptamer conjugate can be configured to provide 234,000 Lux intensity at 505 nm.

In some embodiments, the step of blocking or filtering out the light includes using one or more filters, wherein the one or more filters are configured to substantially block the excitation wavelength(s) while allowing substantially all of the emission wavelength(s) to pass through for detection and/or visualization and/or imaging. In some embodiments, the filter is between the surface and the viewer, a detection device, or an imaging device to aid for visualization of fluorescence emitted from the surface and/or improve the signal to noise ratio. Depending on the fluorophore(s) used, different filters can be used for optimal visualization. For example, if the different fluorophores have sufficiently different emission wavelengths different filters might be required for optimal visualization. In some embodiments, visualization can be performed in ambient light or in a dark environment.

In some embodiments, the filter is a bandpass filter, a long pass filter or a combination thereof. Bandpass filters allow a band of light to pass through the filter to the detector (for example, a bandpass at 580 nm allows light from about 555 nm-605 nm to pass through). Long pass filters allow light at a higher wavelength to pass through to the detector (for example, a long pass at 580 nm allows light from about 580 nm-end of visible spectrum).

The method according to some embodiments comprises a set of conditions for illuminating the location using a lighting device as described herein. In some embodiments, the lighting device comprises a single emitter and the lighting device is configured to allow switching between two or more different wavelengths, each wavelength being suited to a specific detectable molecule (e.g. fluorophore) and/or interchangeable filter.

In some embodiments, the lighting device is a portable lighting device. In some embodiments, the lighting device is a handheld lighting device. In some embodiments, the lighting device is a cordless device. In some embodiments, the lighting device is battery operated and rechargeable. In some embodiments, the battery is integrated into the device. In some embodiments, the battery is removable. Non limiting lighting devices include flashlight, torch, and the like.

Lighting devices are well known and available to persons of skill in the art. In some embodiments, the lighting device may be in the form of a battery operated, handheld LED light source (e.g. available from e.g. Rofin Forensic). For example, the lighting device is a Polilight-Flare® Plus 2 having an output ranging from 485 nm to 515 nm with a peak wavelength at 505 nm to allow for example visualization of FAM-aptamer conjugates.

In some embodiments, the lighting device is NightSea Xite Royal Blue (XRB) which emits 440-460 nm, NightSea prototype Blue light which emits 470-490 nm or NightSea Cyan (CY) which emits 490-515 nm to allow for example visualization of FAM-aptamer conjugates.

In some embodiments, the lighting device is custom made to emit at an exact wavelength of 485 nm having a +/−2% variance.

In some embodiments, the method further comprises imaging (e.g. photographing) the location and detecting the presence or absence of the target molecule (or pathogens).

In some embodiments, the method may comprise the use of a bandpass filter in combination with the lighting device. The bandpass filter may be configured to transmit light of a certain wavelength band and reject stray light outside the predetermined wavelength band. In some embodiments, the bandpass filter is a 590 nm bandpass filter allowing light from 570 to 610 nm to pass through to the detector.

In some embodiments, the method may comprise the use of a longpass filter in combination with the lighting device. Longpass filters transmit wavelengths of light longer than or above the designated wavelength and block or reject light at shorter wavelengths. In some embodiments, the longpass filter is 550 nm blocking light shorter than 550 nm but allowing longer wavelengths of light pass through to the detector.

In some embodiments, the one or more filters may be worn as eyewear, for example goggles and eyeglasses. In other embodiments, the filter may be a feature of the imaging device (for example a camera) used to image the surface.

In some embodiments, the step of visualizing the location may be performed under ambient light or in dark conditions.

In some embodiments, the method may further comprise visualizing the location with viewing goggles, glasses, or the like. In some embodiments, the goggles are orange and thus are suitable for use in combination with a lighting device which produces light having a wavelength of between about 485 nm-515 nm, e.g. 505 nm, and an aptamer which comprises a detection molecule that emits at a wavelength of approximately 505 nm.

In some embodiments, the fluorescence can be observed visually. In some embodiments, the fluorescence can be observed with an instrument. In some embodiments, the instrument is a fluorometer. In some embodiments, the fluorometer can be calibrated so as to allow for the observation of the signal at a predetermined wavelength and/or at a predetermined intensity.

### Kits

Embodiments also provide a kit for detecting and/or quantifying target molecules, wherein the kit comprises one or more aptamers associated with a detectable molecule as described herein.

Embodiments provide a kit that further comprises a lighting device as described herein. In some embodiments, the kit may further comprise a bandpass or a long pass filter as described herein. In some embodiments, the kit may comprise viewing goggles or glasses or the like as described herein. In some embodiments, the kit may comprise a spraying device or the like as described herein.

In some embodiments, the kit comprises:
a) A solution comprising an aptamer or a cocktail of aptamers having one or more fluorophore capable of emitting at one or more emission wavelengths;
b) A lighting device configured to emit light at one or more predetermined excitation wavelengths;
c) One or more filter (bandpass or long pass filter)
d) A spraying device

In some embodiments, the kit further comprises viewing eyewear comprising the bandpass or the long pass filter.

In some embodiments, the kit further comprises a polarizing filter. Polarizing filters can be used to reduce glare or reflection of light coming from surfaces (e.g. glass or metal). In some embodiments, the polarizing filter is a circular polarizing filter. In some embodiments, the polarizing filter can be part of an imaging device. For example, a circular polarizing filter can be attached to the lens of a camera.

For example, the kit comprises:
a) A solution comprising aptamers having a detection molecule conjugated thereto e.g. a fluorophore capable of emitting at a wavelength of between about 485-516 nm. In some embodiments, the fluorophore is capable of emitting at a wavelength with a peak of 517 and being excited at a peak of between about 490-505 nm. In some embodiments, the fluorophore is capable of being excited at a peak of 495 nm.
b) A lighting device. In some embodiments, the lighting device is configured to emit light having a wavelength of between about 485-515 nm. In some embodiments, the lighting device is configured to emit light having a wavelength of between about 490-505 nm;
c) A filter. In some embodiments, the filter comprises or consist of a 590 nm bandpass filter; in some embodiments, the filter is 550 nm longpass filter;
d) Viewing goggles. In some embodiments, the viewing goggles are orange viewing goggles. In some embodiments, the viewing goggles are a bandpass filter. In some embodiments, the viewing goggles are a longpass filter.
e) Polarizing filter. In some embodiments, the polarizing filter is a circular polarizing filter to be used in imaging the surface.
f) Spraying device. In some embodiments, the spraying device is a manual sprayer. In some embodiments, the spraying device has aerosol functionality. In some embodiments, the spraying device is an atomizer sprayer. In some embodiments, the spraying device is a fogger sprayer.

In some embodiments, the kit further comprises instructions for use in accordance with any of the methods described herein.

The kit may comprise further components for the reaction intended by the kit or the method to be carried out, for example components for an intended detection of enrichment, separation and/or isolation procedures. Non-limiting examples include buffer solutions, substrates for a color reaction, dyes, or enzymatic substrates. In the kit, the aptamer may be provided in a variety of forms, including but not limited to being pre-immobilized onto a support (e.g. solid support), freeze-dried, or in a liquid medium.

A kit herein may be used for carrying out any method described herein. It will be appreciated that the parts of the kit may be packaged individually in vials or in combination in containers or multi-container units. Typically, manufacture of the kit follows standard procedures which are known to the person skilled in the art.

### Uses

In some embodiments, method of detecting target molecule using conjugated aptamers is provided. The method may comprise interacting the sample with a conjugated aptamer described herein and detecting the presence, absence, and/or amount of one or more target molecules (of one or more pathogen). The method may be for detecting the presence, absence, and/or amount of the target molecule in a sample using a detection method including, but not limited to, photonic detection, electronic detection, acoustic detection, electrochemical detection, electro-optic detection, enzymatic detection, chemical detection, biochemical detection, or physical detection.

In some embodiments, the method is for detecting the presence, absence, or amount of one or more target molecule, on a surface. In some embodiments, the aptamers and method provided may have utility in detecting pathogens on surfaces in hospital and healthcare facilities. Non-limiting examples of surfaces may include bed linen, medical equipment, clothing, floors, walls, and the like.

In some embodiments, the method may be for use in detecting target molecules, in a sample previously obtained from a surface as described herein.

In some embodiments, the methods of the disclosure may be used to detect surface proteins, antigen etc. . . . In some embodiments, the methods of the disclosure may be used to detect pathogens (including but not limited to C. difficile).

In some embodiments, the methods may be used to detect one or more target surface proteins or one or more pathogens in real-time. Following detection and/or quantification of the one or more pathogens, action may be taken to kill and/or remove the pathogens. Non-limiting examples of such action may include washing or destruction of bed linen, and/or cleaning of surfaces including but not limited to medical equipment, beds, walls, floors, and the like. Measures such as isolation of patients and enforcement of stringent hygiene protocols may also be taken.

In an aspect of the present disclosure, there is provided a composition comprising at least one aptamer, wherein at least one of the aptamers is as described herein wherein the composition optionally comprises at least one of water, salts, one or more buffer herein, a detergent, and BSA. In an aspect of the present disclosure, there is provided a composition comprising at least one aptamer and graphene oxide, wherein at least one of the aptamers is as described herein wherein the composition optionally comprises at least one of water, salts, one or more buffer herein, a detergent, and BSA. In some embodiments, the composition is a solution. In an aspect of the present disclosure, there is provided a composition comprising at least one aptamer and optionally comprises at least one of water, salts, one or more buffer herein, a detergent, and BSA.

In an aspect of the present disclosure, there is provided an apparatus for detecting the presence, absence, or level of surface antigen in a sample, the apparatus comprising:
i. a support; and
ii. an aptamer as described herein.

In embodiments, the apparatus is for detecting the presence, absence or level of surface antigen in a sample.

In embodiments, the sample can be a sample previously obtained from a subject suspected of having or diagnosed with a bacterial or viral infection. In embodiments, the sample can be an object located in a hospital environment, for example bedding, furniture, building structures.

In embodiments, the support is a bead, a microtiter or other assay plate, a strip, a membrane, a film, a gel, a chip, a microparticle, a nanoparticle, a nanofiber, a nanotube, a micelle, a micropore, a nanopore or a biosensor surface.

In embodiments, the apparatus is suitable for surface plasmon resonance (SPR), biolayer interferometry (BLI), lateral flow assay and/or enzyme-linked oligonucleotide assay (ELONA).

In an aspect of the present disclosure, there is provided a use of an aptamer a complex, a biosensor or test strip, a composition or apparatus as described herein for detecting, enriching, separating and/or isolating surface antigens.

In an aspect of the present disclosure, there is provided a method of detecting the presence, absence, or amount of surface antigens in a sample, the method comprising: interacting the sample with an aptamer, a complex, or a composition as described herein; and detecting the presence, absence or amount of surface antigens.

In some embodiments, the method is for detecting the presence, absence, or amount of surface proteins in a sample.

In some embodiments, the presence, absence, or amount of surface antigens is detected by photonic detection, electronic detection, acoustic detection, electrochemical detection, electro-optic detection, enzymatic detection, chemical detection, biochemical detection or physical detection.

In an aspect of the present disclosure, there is provided a kit for detecting and/or quantifying surface antigens, the kit comprising an aptamer as described herein.

### EXAMPLES

In the following, the disclosure will be explained in more detail by means of non-limiting examples of specific embodiments. In the example experiments, standard reagents and buffers free from contamination are used.

### Example 1: Visualization of Pathogens on Surface of Hospital Room

Surfaces (non-limiting examples of surfaces are doorknobs, bedrails, chair armrests, sink faucet knobs) in a patient hospital room were analyzed for contamination by pathogen(s) of interest, for example C. difficile. The identified surface(s) were sprayed with a solution containing FAM-aptamer conjugate and graphene oxide whereby the surface is covered with a thin, uniform deposition of solution. The solution dried within minutes and visualization took place by passing a forensic torch (Polilight-Flare® Plus 2 Cyan light having an output ranging from 485 nm to 515 nm with a peak wavelength at 505 nm) over the dried surface. Areas of pathogen contamination was determined by the user wearing appropriate filter glasses.

### Example 2: Visualization of Pathogens on Surface of Hospital Room

Surfaces in a patient hospital room are analyzed for contamination by pathogen(s) of interest, for example C. difficile. The identified surface(s) are sprayed with a solution containing Cy3-aptamer conjugate and graphene oxide whereby the surface is covered with a thin, uniform deposition of solution. The solution dries within minutes and visualization took place by passing a forensic torch (NIGHTSEA Cyan light (having an output ranging from 490 to 515 nm). Alternatively, Poliflare “green” light (having an output ranging from 510 to 545 nm with a peak wavelength at 530 nm) can be used. Areas of pathogen contamination was determined by the user wearing appropriate filter glasses.

The references cited throughout this application, are incorporated herein in their entireties for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this disclosure is not limited to the particular embodiments disclosed but is intended to cover all modifications which are within the spirit and scope of the disclosure as defined by the appended claims; the above description; and/or shown in the attached drawings.

The invention claimed is:

1. A method of visualizing presence of one or more pathogen on a surface, comprising:
a. applying a substantially uniform layer of a liquid composition to the surface, the liquid composition comprising:
(i) one or more aptamer conjugate, the one or more aptamer conjugate including one or more aptamer covalently or non-covalently associated with one or more fluorophore, wherein the one or more aptamer conjugate has a specific binding affinity for one or more target molecule of the one or more pathogens; and
(ii) a quencher to quench nonspecific fluorescence;
b. exposing the surface to a predetermined temperature over a period of time so as to allow a population of conjugated aptamers to bind to the one or more target molecule;
c. illuminating at least one area of the surface by emitting a light at one or more predetermined wavelength range at or around an excitation maximum wavelength of the one or more fluorophore, wherein the one or more fluorophore absorbs the emitted light at the one or more predetermined wavelength range and emits light at a longer wavelength range; and
d. blocking the emitted light, wherein the step of blocking blocks (i) the one or more excitation wavelength range and transmits a second wavelength range that includes some or all of the wavelengths emitted by the one or more fluorophore; or (ii) wavelengths shorter than the emitted wavelength range of the fluorophore.

2. The method of claim 1, wherein the step of applying comprises spraying the surface with the liquid composition or atomizing the liquid composition onto the surface, wherein the spraying or the atomizing comprises dispersing droplets onto the surface, wherein the droplets have an average size ranging from about 10 to about 100 mm.

3. The method of claim 2, wherein the step of spraying includes a spray distribution with a coefficient of variation (CV) of less than 15%.

4. The method of claim 2, wherein the spraying includes minimizing disturbance of the one or more pathogen on the surface.

5. (canceled)

6. (canceled)

7. The method of claim 1, wherein step (b) comprises incubating the surface with the one or more aptamer conjugate at room temperature for about 1 min to about 10 min or for about to 1 min to less than 5 min.

8. (canceled)

9. The method of claim 1, wherein the liquid composition comprises two or more conjugated aptamers, wherein each of the two or more aptamer is conjugated to a different fluorophore.

10. The method of claim 9, wherein the step of illuminating comprises emitting the light at two or more predetermined wavelength range at or around the excitation maximum wavelength of two or more of the different fluorophores.

11. The method of claim 1, wherein the step of illuminating comprises emitting the light at an illumination intensity to enable visualization of the fluorophore at the one or more predetermined wavelength range at or around the excitation maximum wavelength of the one or more fluorophore.

12. The method of claim 1, wherein the step of blocking the light comprises using one or more filter, wherein the one or more filter comprises a long pass filter.

13. The method of claim 1, wherein the quencher comprises or consists of graphene oxide.

14. The method of claim 1, wherein the one or more target molecule comprises a protein.

15. The method of claim 1, wherein the liquid composition comprises two or more different aptamers, and wherein the two or more different aptamers bind to different target proteins or wherein the two or more different aptamers bind to a same target protein.

16. (canceled)

17. (canceled)

18. The method of claim 1, the method further comprising imaging the surface.

19. The method of claim 1, the method further comprising identifying the one or more pathogen.

20. The method of claim 1, the method further comprising decontaminating the surface.

21. The method of claim 20, the method further comprising (e) determining the presence of fluorescence from the population of conjugated aptamers or absence of fluorescence thereby determining the presence or absence of the one or more pathogens onto the surface.

22. The method of claim 20, the method further comprising repeating steps (a) through (e).

23. The method of claim 1, wherein in step (b) a first population of conjugated aptamers binds to the one or more target; and a second population of conjugated aptamers does not bind to the one or more target molecule and remains associated with the quencher.

24. The method of claim 1, wherein step (c) comprises illuminating the at least one area of the surface with a lighting device having a single emitter that is configured to emit the light at the one or more predetermined wavelength range at or around the excitation maximum wavelength of the one or more fluorophore.

25. The method of claim 24, wherein the emitter is configured to emit the light at two or more predetermined wavelength range at or around the excitation maximum wavelength of two or more of different fluorophores.
